(19) United States
(12) Patent Application Publication
(10) Pub. No.: US 2025/0182849 A1
(43) Pub. Date: Jun. 5, 2025

(54) SINGLE NUCLEOTIDE POLYMORPHISM (SNP) MARKER FOR POLYGENIC RISK SCORE (PRS) OF CHRONIC OBSTRUCTIVE PULMONARY DISEASE (COPD) AND USE THEREOF, AND DEVICE

(71) Applicant: Institute of Basic Medical Sciences, CAMS, Beijing (CN)

(72) Inventors: Jing WANG, Beijing (CN); Junling PANG, Beijing (CN); Erping LONG, Beijing (CN)

(73) Assignee: Institute of Basic Medical Sciences, CAMS, Beijing (CN)

(21) Appl. No.: 18/630,255

(22) Filed: Apr. 9, 2024

(30) Foreign Application Priority Data
Dec. 4, 2023 (CN) 202311648483.1

(51) Int. Cl. G16B 20/20; G16H 50/30

(52) U.S. Cl. CPC G16B 20/20; G16H 50/30

(57) ABSTRACT

The present disclosure provides a single nucleotide polymorphism (SNP) marker for a polygenic risk score (PRS) of chronic obstructive pulmonary disease (COPD) and use thereof, and a device, and relates to the technical field of medical detection. In the present disclosure, the SNP marker for a PRS of COPD is provided for the first time, and a calculation formula for the PRS of the COPD is provided to calculate information of the SNP marker, thereby obtaining a genetic risk score (GRS) of the COPD, namely the PRS. The SNP marker realizes risk stratification of the COPD in an East Asian population, especially in a Chinese population. The present disclosure further provides for the first time a device for detecting, calculating, and displaying the GRS of the COPD.

### CROSS REFERENCE TO RELATED APPLICATION

This patent application claims the benefit and priority of Chinese Patent Application No. 2023116484831, filed with the China National Intellectual Property Administration on Dec. 4, 2023, the disclosure of which is incorporated by reference herein in its entirety as part of the present application.

### TECHNICAL FIELD

The present disclosure relates to the technical field of medical detection, and specifically relates to a single nucleotide polymorphism (SNP) marker for a polygenic risk score (PRS) of chronic obstructive pulmonary disease (COPD) and use thereof, and a device.

### BACKGROUND

Chronic obstructive pulmonary disease (COPD) is a respiratory disease with high morbidity and mortality, characterized by persistent respiratory symptoms and airway limitation. Among the top 10 causes of death in the world in 2019 announced by the World Health Organization (WHO), COPD is the third leading cause of death, accounting for approximately 6% of the total deaths. The number of COPD patients in China is close to 100 million. Due to long illness cycle, easy recurrence of acute exacerbations, and multiple comorbidities, the COPD not only seriously affects a quality of life of patients, but also imposes a heavy medical and economic burden on the entire society.

COPD is a complex polygenic disease, and its occurrence is believed to be the result of a combination of genetic factors and environmental factors (such as smoking). In recent years, studies using family and population cohorts have found that there are considerable loci associated with susceptibility to COPD in the genome. These susceptibility loci can be adopted to conduct risk assessment on the population and provide first-level early warning to people with high genetic risk, so as to improve their unhealthy lifestyle. This process has important guiding significance for reducing the incidence of COPD and assisting early diagnosis and treatment.

Polygenic risk score (PRS), sometimes also called genetic risk score (GRS), is mainly used to assess the genetic risk of an individual suffering from a certain disease, and serves as a research content with broad research value and application prospects. The specific implementation of PRS relies on two key elements: (1) genetic variation of an individual, generally a single nucleotide polymorphism (SNP); (2) an effect value of the genetic variation (generally BETA or odds ratio (OR)), which is mainly derived from large-scale genome wide association study (GWAS). The GRS of an individual suffering from the disease is obtained through a specific calculation formula. Generally, a high PRS means that the individual is more likely to suffer from the disease in future life, suggesting the demand to pay more attention to prevention.

To date, almost all large cohort studies and GRS of the COPD have been based on European population cohorts. However, some studies have shown that there are differences in variation locus frequencies among different populations, and there are differences in linkage disequilibrium patterns. These differences result in the GRS of European populations not being used in East Asian populations. Therefore, GWAS information on COPD in East Asian populations in conducting GRS is of great significance for achieving risk stratification, primary warning, and early prevention of COPD in East Asian populations.

### SUMMARY

In view of this, an objective of the present disclosure is to provide an SNP marker for a PRS of COPD. A GRS of the COPD (namely the PRS) is calculated based on information of the SNP marker, thereby achieving risk stratification of the COPD in an East Asian population.

To solve the above technical problems, the present disclosure provides the following technical solutions:

The present disclosure provides an SNP marker for a PRS of COPD, including any one or more of the following SNPs: rs9391855, rs1246642, rs7676488, rs1529672, rs2161245, rs4803402, rs11878604, rs2288450, rs2456020, rs7295442, rs77768175, rs11066132, rs11066015, rs79105258, rs12229654, and rs141965732.

The present disclosure further provides an SNP marker for a PRS of COPD, further including any one or more of the following SNPs in addition to the SNP marker described above: rs7024396, rs877116, rs1859788, rs9272466, rs3104376, rs1931982, rs9496212, rs2877162, rs11168049, rs2057656, rs10036896, rs6839086, rs13143549, rs12509944, rs4860797, rs13141641, rs4408914, rs769671, rs879394, rs6062899, rs75550771, rs62201158, rs17409597, rs7598305, rs4851569, rs2160203, rs383925, rs7251570, rs7504262, rs62065216, rs4795400, rs11658786, rs34898535, rs12050525, rs11854507, rs12905273, rs11072790, rs11072774, rs8040868, rs28669908, rs7359276, rs8042849, rs2009746, rs2568485, rs28523913, rs12908092, rs7181877, rs12232354, rs7132778, rs12579396, rs12231873, rs76579145, rs75295329, rs7130588, rs10495098, rs2099684, and rs11205303.

The present disclosure further provides use of the SNP marker in preparation of a product for assessing an individual COPD risk.

Preferably, a PRS value is calculated according to a PRS formula of the COPD, and the PRS value is compared with a genetic risk stratification table to determine the individual COPD risk;
the PRS formula of the COPD is:

$${PRS}_{j} = ( {\sum }_{i}^{N} {β}_{i} \times {G}_{ij} ) / ( P \times {M}_{j} )$$

PRSⱼ represents the PRS of a j-th tested individual; N represents a total number of the SNPs included in PRS calculation; βᵢ represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j;
when the SNP marker includes one or more of the above 16 SNPs (rs9391855, rs1246642, rs7676488, rs1529672, rs2161245, rs4803402, rs11878604, rs2288450, rs2456020, rs7295442, rs77768175, rs11066132, rs11066015, rs79105258, rs12229654, and rs141965732), the genetic risk stratification table is shown in Table 1:

TABLE 1
Genetic risk stratification table of 16 SNPs

| | | | | | |
|---|---|---|---|---|---|
| PRS | <−0.0205 | −0.0205 | −0.0132 | −0.005 to | >0.0136 |
| | | to −0.0132 | to −0.005 | 0.0136 | |
| Risk grouping | Low | Mid-low | Moderate | Mid-high | High |

when the SNP marker includes one or more of the following 57 SNPs (rs7024396, rs877116, rs1859788, rs9272466, rs3104376, rs1931982, rs9496212, rs2877162, rs11168049, rs2057656, rs10036896, rs6839086, rs13143549, rs12509944, rs4860797, rs13141641, rs4408914, rs769671, rs879394, rs6062899, rs75550771, rs62201158, rs17409597, rs7598305, rs4851569, rs2160203, rs383925, rs7251570, rs7504262, rs62065216, rs4795400, rs11658786, rs34898535, rs12050525, rs11854507, rs12905273, rs11072790, rs11072774, rs8040868, rs28669908, rs7359276, rs8042849, rs2009746, rs2568485, rs28523913, rs12908092, rs7181877, rs12232354, rs7132778, rs12579396, rs12231873, rs76579145, rs75295329, rs7130588, rs10495098, rs2099684, and rs11205303) in addition to the above 16 SNPs, the genetic risk stratification table is shown in Table 2:

TABLE 2
Genetic risk stratification table of above 16 SNPs

| | | | | | |
|---|---|---|---|---|---|
| PRS | <0.0054 | 0.0054 | 0.003 to 0 | 0 to | >0.0043 |
| | | to 0.003 | | 0.0043 | |
| Risk grouping | Low | Mid-low | Moderate | Mid-high | High. |

Preferably, the individual is an individual from an East Asian population.

The present disclosure further provides a device for assessing an onset risk of COPD, including a detection unit, a data analysis unit, and a display; where the detection unit is configured to detect locus information of the SNP marker in an individual.

Preferably, the detection unit detects the SNP information of a sample to be tested by the following methods: multiplex PCR-targeted amplicon sequencing, MassARRAY nucleic acid mass spectrometry, customized SNP chip detection, or high-throughput sequencing.

Preferably, the data analysis unit includes a data receiving module, a data analysis module, and an output module; the data analysis module is configured to determine an effect value and a number of effect alleles according to a detection result of the detection unit corresponding to the following Table 3, and then execute the PRS formula of the COPD to obtain the PRS value,

TABLE 3
Effect values and number of effect
alleles at corresponding SNP loci

| | | Chromo- | | Non- | Effect value |
|---|---|---|---|---|---|
| | Chromo- | some | Effect | effect | of the effect |
| SNP | some | position | allele | allele | allele |
| rs9391855 | 6 | 32182024 | T | C | −0.1705 |
| rs1246642 | 4 | 88943295 | T | C | 0.1446 |
| rs7676488 | 4 | 7889408 | T | C | 0.1131 |
| rs1529672 | 3 | 25479091 | A | C | −0.1456 |
| rs2161245 | 19 | 40915612 | A | G | 0.1852 |
| rs4803402 | 19 | 40906280 | A | G | 0.1279 |
| rs11878604 | 19 | 40827379 | T | C | 0.1319 |
| rs2288450 | 19 | 40703272 | T | C | −0.1908 |
| rs2456020 | 15 | 78576056 | T | C | −0.1402 |
| rs7295442 | 12 | 112548943 | T | G | −0.1386 |
| rs77768175 | 12 | 112298314 | A | G | −0.2109 |
| rs11066132 | 12 | 112030402 | T | C | 0.2117 |
| rs11066015 | 12 | 111730205 | A | G | 0.2036 |
| rs79105258 | 12 | 111280427 | A | C | 0.1848 |
| rs12229654 | 12 | 110976657 | T | G | −0.1852 |
| rs141965732 | 12 | 110144533 | T | C | 0.1961 |
| rs7024396 | 9 | 125892044 | T | C | 0.0482 |
| rs877116 | 8 | 10855435 | T | G | −0.0366 |
| rs1859788 | 7 | 100374211 | G | A | −0.0391 |
| rs9272466 | 6 | 32637919 | T | G | 0.0766 |
| rs3104376 | 6 | 32632226 | C | T | 0.0429 |
| rs1931982 | 6 | 142235574 | T | C | −0.0363 |
| rs9496212 | 6 | 141978208 | A | G | −0.0381 |
| rs2877162 | 5 | 44415290 | G | T | 0.0411 |
| rs11168049 | 5 | 148475701 | C | T | −0.0337 |
| rs2057656 | 5 | 132473613 | C | T | −0.0469 |
| rs10036896 | 5 | 132449486 | T | C | 0.0467 |
| rs6839086 | 4 | 7869528 | T | G | −0.0584 |
| rs13143549 | 4 | 66944813 | G | A | −0.0391 |
| rs12509944 | 4 | 66936710 | C | T | −0.0344 |
| rs4860797 | 4 | 66935416 | A | G | −0.0367 |
| rs13141641 | 4 | 144585304 | C | T | −0.0603 |
| rs4408914 | 4 | 144324990 | T | G | −0.0372 |
| rs769671 | 4 | 139962464 | T | C | −0.0369 |
| rs879394 | 3 | 168992055 | T | G | 0.0401 |
| rs6062899 | 20 | 63348441 | A | G | −0.0634 |
| rs75550771 | 2 | 55839380 | G | A | 0.1269 |
| rs62201158 | 2 | 228688181 | G | A | −0.0635 |
| rs17409597 | 2 | 145287492 | C | T | 0.0393 |
| rs7598305 | 2 | 144884850 | T | G | 0.0386 |
| rs4851569 | 2 | 102366787 | A | C | 0.0353 |
| rs2160203 | 2 | 102344364 | G | A | −0.0414 |
| rs383925 | 19 | 54279666 | C | T | −0.0512 |
| rs7251570 | 19 | 40835845 | G | A | 0.0392 |
| rs7504262 | 18 | 37580180 | A | G | 0.0349 |
| rs62065216 | 17 | 40062520 | A | G | 0.0352 |
| rs4795400 | 17 | 39910767 | T | C | −0.0417 |
| rs11658786 | 17 | 39659646 | A | G | −0.0343 |
| rs34898535 | 16 | 31014320 | T | C | −0.0427 |
| rs12050525 | 15 | 78783404 | C | T | 0.0483 |
| rs11854507 | 15 | 78776779 | G | A | 0.0534 |
| rs12905273 | 15 | 78710413 | G | A | −0.0415 |
| rs11072790 | 15 | 78699683 | T | C | 0.0535 |
| rs11072774 | 15 | 78660355 | T | C | −0.0503 |
| rs8040868 | 15 | 78618839 | C | T | 0.0974 |
| rs28669908 | 15 | 78617925 | A | C | −0.0866 |
| rs7359276 | 15 | 78600319 | T | C | 0.0799 |
| rs8042849 | 15 | 78525587 | T | C | −0.0868 |
| rs2009746 | 15 | 78461760 | G | A | 0.0856 |
| rs2568485 | 15 | 78459772 | T | C | 0.0712 |
| rs28523913 | 15 | 77900910 | T | C | 0.0406 |
| rs12908092 | 15 | 71380414 | C | T | 0.0442 |
| rs7181877 | 15 | 67184397 | A | G | −0.0470 |
| rs12232354 | 15 | 49351432 | T | C | −0.0653 |
| rs7132778 | 12 | 112503550 | A | C | 0.0846 |
| rs12579396 | 12 | 112157010 | T | C | 0.0813 |
| rs12231873 | 12 | 111894111 | T | C | 0.0831 |
| rs76579145 | 12 | 111629113 | T | C | 0.0709 |
| rs75295329 | 12 | 110906817 | T | G | 0.1160 |
| rs7130588 | 11 | 76559639 | G | A | 0.0363 |
| rs10495098 | 1 | 218342968 | T | G | 0.0346 |
| rs2099684 | 1 | 161530340 | G | A | 0.0422 |
| rs11205303 | 1 | 149934520 | C | T | 0.0378. |

Preferably, the output module is configured to compare the PRS value obtained by the data analysis module with the genetic risk stratification table in Table 1 or 2, and then determine the individual COPD risk.

Preferably, the display is connected to the output module and then configured to display results of the detection unit, the data analysis module, and the output module.

The present disclosure has following beneficial effects:

In the present disclosure, the SNP marker for a PRS of COPD is provided for the first time, and a calculation formula for the PRS of the COPD is provided. The calculation formula for the PRS of the COPD is adopted to calculate information of the SNP marker, thereby obtaining a genetic risk score (GRS) of the COPD, namely the PRS. The SNP marker realizes risk stratification of the COPD in an East Asian population, especially in a Chinese population. The SNP marker provided by the present disclosure can conduct the risk assessment of COPD on a sample to be tested with a desirable accuracy.

The present disclosure further provides for the first time a device for detecting, calculating, and displaying the GRS of the COPD. The device presents a final scoring result through a display, and can easily and quickly realize the risk assessment of COPD to well assess the onset risk of COPD in the East Asian population. Therefore, the device is of great significance for risk warning and primary prevention of the COPD.

### BRIEF DESCRIPTION OF THE DRAWINGS

FIG. 1A shows a process of obtaining 16 SNPs, where FIG. 1A is the data source and analysis process on the GWAS results of a Chinese CPH cohort and the GWAS results of a Japanese BBJ cohort;

FIG. 2 shows the effect of 61 SNPs selected from a public database;

FIGS. 3A-3C show the validation results of a PRS scoring system using 73 SNPs in an independent population cohort, where FIG. 3A is a receiver operating characteristic (ROC) curve for an accuracy of predicting COPD in the population; FIG. 3B is an odds ratio (OR) of the proportion of sick people in other intervals relative to the first interval after PRS scores are divided from low to high; and FIG. 3C is a proportion of the number of sick people and the number of non-sick people in each interval after the PRS value is divided from low to high; and

FIGS. 4A-4C show the validation results of a PRS scoring system using 16 SNPs in an independent population cohort, where FIG. 4A is an ROC curve for an accuracy of predicting COPD in the population; FIG. 4B is an OR of the proportion of sick people in other intervals relative to the first interval after PRS scores are divided from low to high; and FIG. 4C is a proportion of the number of sick people and the number of non-sick people in each interval after the PRS value is divided from low to high.

### DETAILED DESCRIPTION OF THE EMBODIMENTS

The present disclosure provides an SNP marker for a PRS of COPD, including any one or more of the following 16 SNPs: rs9391855, rs1246642, rs7676488, rs1529672, rs2161245, rs4803402, rs11878604, rs2288450, rs2456020, rs7295442, rs77768175, rs11066132, rs11066015, rs79105258, rs12229654, and rs141965732. More preferably, all of the above 16 SNPs are used. The 16 core SNPs are derived from a meta-analysis of the GWAS of COPD in a Chinese population cohort and the GWAS of COPD in a Japanese population cohort, and mainly reflect the susceptibility locus characteristics of COPD in the East Asian population.

The present disclosure further provides an SNP marker for a PRS of COPD, further including any one or more of the following 57 SNPs in addition to the 16 SNP marker described above: rs7024396, rs877116, rs1859788, rs9272466, rs3104376, rs1931982, rs9496212, rs2877162, rs11168049, rs2057656, rs10036896, rs6839086, rs13143549, rs12509944, rs4860797, rs13141641, rs4408914, rs769671, rs879394, rs6062899, rs75550771, rs62201158, rs17409597, rs7598305, rs4851569, rs2160203, rs383925, rs7251570, rs7504262, rs62065216, rs4795400, rs11658786, rs34898535, rs12050525, rs11854507, rs12905273, rs11072790, rs11072774, rs8040868, rs28669908, rs7359276, rs8042849, rs2009746, rs2568485, rs28523913, rs12908092, rs7181877, rs12232354, rs7132778, rs12579396, rs12231873, rs76579145, rs75295329, rs7130588, rs10495098, rs2099684, and rs11205303. More preferably, all the above 16 SNPs and all the above 57 SNPs are used, that is, a total of 73 SNPs are used. The 57 SNPs are derived from a meta-analysis of previously reported multiple population cohorts (including European populations and East Asian populations), and represent the common susceptibility locus characteristics of COPD in different ethnic groups.

In the present disclosure, a method for detecting the SNP marker preferably includes: multiplex PCR-targeted amplicon sequencing, MassARRAY nucleic acid mass spectrometry, customized SNP chip detection, or high-throughput sequencing.

The present disclosure further provides use of the SNP marker in preparation of a product for assessing an individual COPD risk.

In the present disclosure, the product preferably includes a kit. During specific use, a PRS value is calculated according to a PRS formula of the COPD, and the PRS value is compared with a genetic risk stratification table to determine the individual COPD risk;
the PRS formula of the COPD is:

$${PRS}_{j} = ( {\sum }_{i}^{N} {β}_{i} \times {G}_{ij} ) / ( P \times {M}_{j} )$$

PRSⱼ represents the PRS of a j-th tested individual; N represents a total number of the SNPs included in PRS calculation; Bi represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j; when the SNP marker includes one or more of the above 16 SNPs, the genetic risk stratification table is shown in Table 1; when the SNP marker includes one or more of the above 57 SNPs in addition to the above 16 SNPs, the genetic risk stratification table is shown in Table 2. In the present disclosure, the individual is preferably an individual from an East Asian population.

The present disclosure further provides a device for assessing an onset risk of COPD, including a detection unit, a data analysis unit, and a display; where the detection unit is configured to detect locus information of the SNP marker in an individual.

In the present disclosure, the detection unit detects the SNP information of a sample to be tested preferably by the following methods: multiplex PCR-targeted amplicon sequencing, MassARRAY nucleic acid mass spectrometry, customized SNP chip detection, or high-throughput sequencing. The detection unit is configured to detect information from the individual to be tested and obtain detection results; and the individual information is preferably the SNP information.

In the present disclosure, the data analysis unit includes preferably a data receiving module, a data analysis module, and an output module; the data analysis module is configured to determine an effect value and a number of effect alleles according to a detection result of the detection unit corresponding to the following Table 3, and then execute the PRS formula of the COPD to obtain the PRS value.

In the present disclosure, the data analysis unit preferably further includes a computer storage medium; and the computer storage medium is configured to receive and store the detection results obtained in the detection unit. The data receiving module can retrieve the detection results from the computer storage medium and input them to a computing module in the matrix form. The output module is preferably configured to compare the PRS value obtained by the data analysis module with the genetic risk stratification table in Table 1 or 2, and then determine the individual COPD risk. The data analysis unit is configured to analyze and process the detection results of the detection unit.

In the present disclosure, the display is connected to the output module and then configured to display results of the detection unit, the data analysis module, and the output module. The display is preferably connected to the output module in the data analysis unit, thereby presenting the results of the data analysis unit and outputting SNP information, PRS, and diagnostic classification results. The display includes data visualization software and a terminal display screen.

In order to make the objectives, technical solutions, and advantages of the present disclosure clearer, the present disclosure will be described in detail below in conjunction with examples, but the examples should not be construed as limiting the protection scope of the present disclosure.

In the following examples, all methods are conventional methods, unless otherwise specified.

All materials and reagents used in the following examples may be commercially available, unless otherwise specified.

### Example 1

A PRS formula for COPD was:

$${PRS}_{j} = ( {\sum }_{i}^{N} {β}_{i} \times {G}_{ij} ) / ( P \times {M}_{j} )$$

PRSⱼ represented the PRS of a j-th tested individual; N represented a total number of the SNPs included in PRS calculation; βᵢ represented an effect value of an effect allele of an i-th SNP; i ranged from 1 to 73; Gᵢⱼ represented a number of the effect alleles of the i-th SNP carried by an individual j, and ranged from 0, 1, or 2; P represented a chromosome ploidy and was 2 in the PRS formula; and Mⱼ represented a number of non-missing SNPs among N SNPs detected in the individual j.

### Example 2

### Acquisition of an SNP Marker

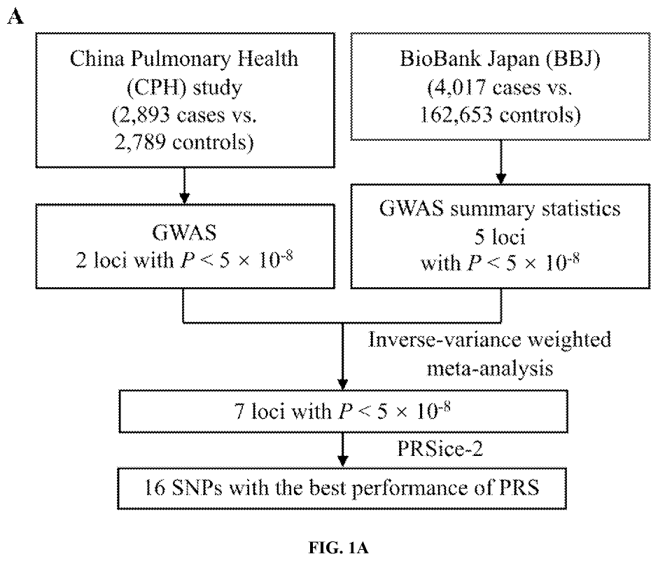

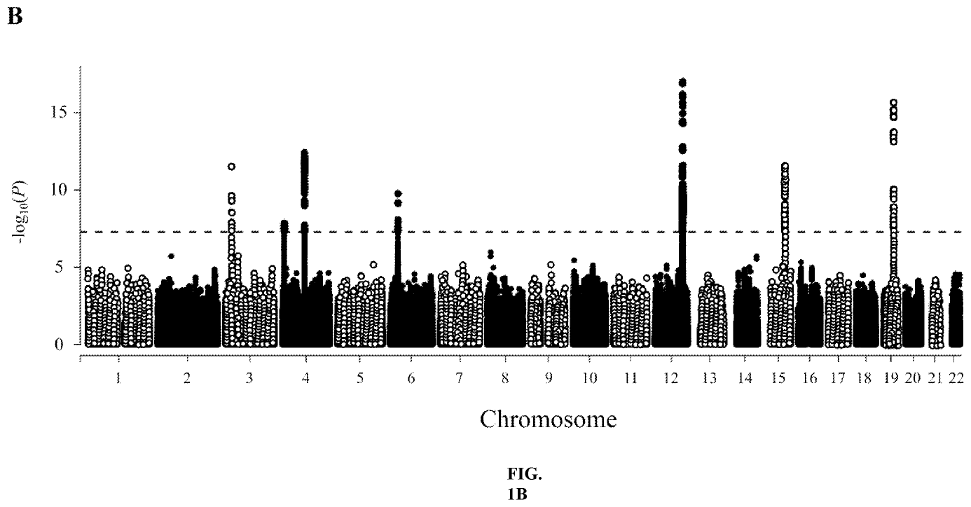

Whole-genome sequencing was conducted on 2,893 COPD patients and 2,789 controls in the China Pulmonary Health (CPH) research project; GWAS was conducted between the obtained SNPs and the phenotype of COPD, and two significant signal regions were obtained. A meta-analysis was conducted based on the published GWAS results (5 significant signals) of 4,017 COPD patients and 162,653 controls in the Japanese BBJ cohort and the results of this application, and a total of 7 significant signal regions were obtained (P<5×10⁻⁸). The generated results were calculated by PRSice-2 software and the PRS scoring effects of independent SNP loci under different P thresholds were comprehensively compared. The calculation principle was the same as the PRS calculation formula in Example 1. Finally, it was found that 16 relatively independent SNP loci under the 5×10⁻⁸ threshold had the optimal PRS effect. The above process was shown in FIG. 1A.

A DNA was extracted from a blood sample of the individual to be tested and submitted to a biological company to allow DNA library construction and sequencing. In this example, the DNA sample of the individual was submitted to Shenzhen WeGene Co., Ltd. for library construction and sequencing. A DNA sequence obtained after sequencing was aligned to a human reference genome GRCh38, and the SNP locus information of the sample was finally obtained through bioinformatics analysis, as shown in Table 4.

TABLE 4
Effect values and number of effect alleles at 16 SNP loci

| SNP | CHR | BP | A1 | A2 | AF1 | BETA | SE |
|---|---|---|---|---|---|---|---|
| rs9391855 | 6 | 32182024 | T | C | 0.1733 | −0.1705 | 0.0267 |
| rs1246642 | 4 | 88943295 | T | C | 0.5865 | 0.1446 | 0.0202 |
| rs7676488 | 4 | 7889408 | T | C | 0.5231 | 0.1131 | 0.0199 |
| rs1529672 | 3 | 25479091 | A | C | 0.3417 | −0.1456 | 0.0209 |
| rs2161245 | 19 | 40915612 | A | G | 0.2771 | 0.1852 | 0.0225 |
| rs4803402 | 19 | 40906280 | A | G | 0.7077 | 0.1279 | 0.0230 |
| rs11878604 | 19 | 40827379 | T | C | 0.6086 | 0.1319 | 0.0204 |
| rs2288450 | 19 | 40703272 | T | C | 0.1489 | −0.1908 | 0.0294 |
| rs2456020 | 15 | 78576056 | T | C | 0.5678 | −0.1402 | 0.0200 |
| rs7295442 | 12 | 112548943 | T | G | 0.3937 | −0.1386 | 0.0219 |
| rs77768175 | 12 | 112298314 | A | G | 0.7710 | −0.2109 | 0.0263 |
| rs11066132 | 12 | 112030402 | T | C | 0.2287 | 0.2117 | 0.0255 |
| rs11066015 | 12 | 111730205 | A | G | 0.2336 | 0.2036 | 0.0237 |
| rs79105258 | 12 | 111280427 | A | C | 0.2505 | 0.1848 | 0.0235 |
| rs12229654 | 12 | 110976657 | T | G | 0.8073 | −0.1852 | 0.0253 |
| rs141965732 | 12 | 110144533 | T | C | 0.1155 | 0.1961 | 0.0349 |

Notes:
CHR represented a chromosome, BP represented a chromosome position; A1 represented the effect allele of the SNP, A2 represented the non-effect allele, and AF1 represented a frequency of the effect allele; BETA represented the effect value of the effect allele, and SE represented a standard deviation of the BETA.

### Example 3

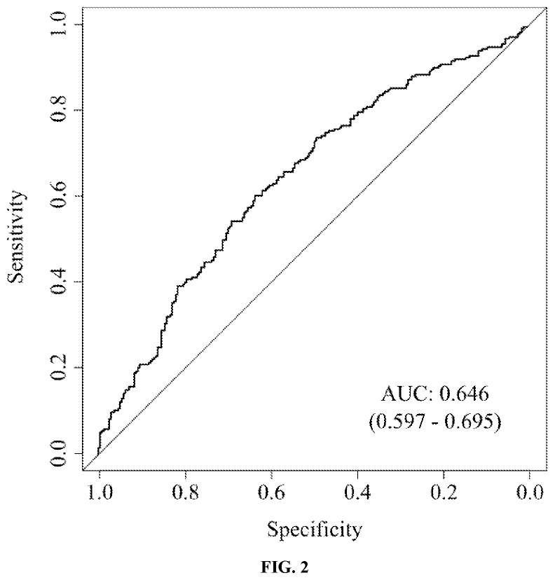

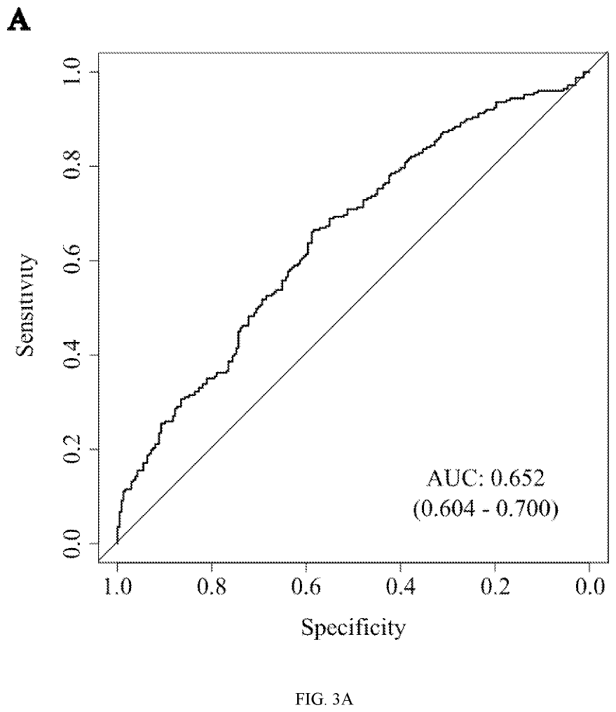

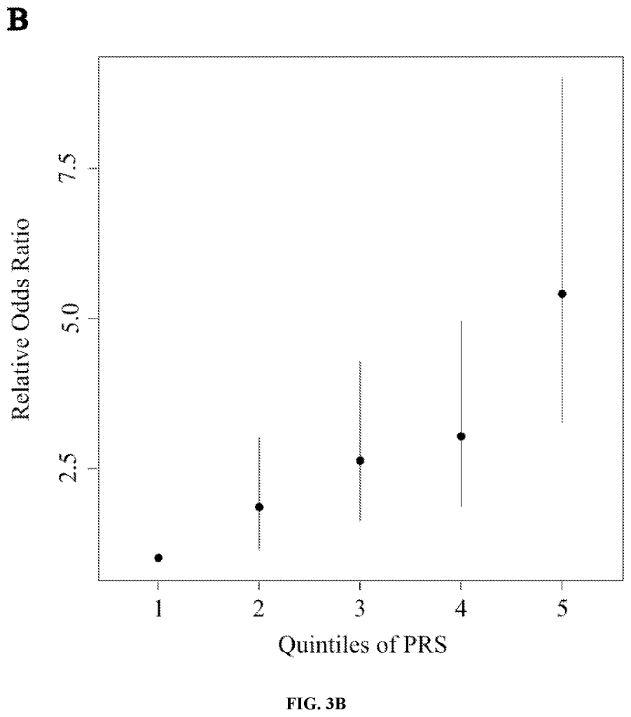

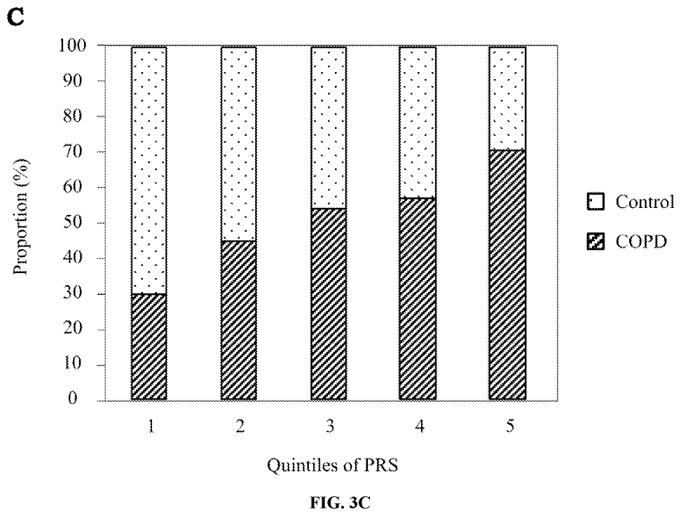

By using multi-ethnic meta-analysis results of the Global Biobank Meta-analysis Initiative (GBMI) (PMID: 36777179), a PRS scoring effect of independent SNP loci under different P thresholds was calculated using the PRSice-2 software according to the PRS calculation formula in Example 1. Finally, it was found that the PRS model constructed with 61 SNPs under the 5×10⁻⁸ threshold showed the optimal effect. As shown in FIG. 2, an area under the curve (AUC) could reach 0.646. There were 4 SNPs as duplicates of the 16 core SNPs in Example 1. After removing these 4 repeated SNPs, 57 expanded SNPs were obtained (Table 5). These 57 expanded SNPs and 16 core SNPs were jointly scored by PRS, and it was found that they had the best effect in predicting COPD (AUC=0.652), as shown in FIG. 3A.

TABLE 5
Effect values and number of effect alleles at 57 SNP loci

| SNP | CHR | BP | A1 | A2 | AF1 | BETA | SE |
|---|---|---|---|---|---|---|---|
| rs7024396 | 9 | 125892044 | T | C | 0.6664 | 0.0482 | 0.0067 |
| rs877116 | 8 | 10855435 | T | G | 0.5085 | −0.0366 | 0.0065 |
| rs1859788 | 7 | 100374211 | G | A | 0.6222 | −0.0391 | 0.0062 |
| rs9272466 | 6 | 32637919 | T | G | 0.2660 | 0.0766 | 0.0103 |
| rs3104376 | 6 | 32632226 | C | T | 0.7902 | 0.0429 | 0.0078 |
| rs1931982 | 6 | 142235574 | T | C | 0.4250 | −0.0363 | 0.0064 |
| rs9496212 | 6 | 141978208 | A | G | 0.4910 | −0.0381 | 0.0069 |
| rs2877162 | 5 | 44415290 | G | T | 0.4021 | 0.0411 | 0.0070 |
| rs11168049 | 5 | 148475701 | C | T | 0.4336 | −0.0337 | 0.0059 |
| rs2057656 | 5 | 132473613 | C | T | 0.7059 | −0.0469 | 0.0069 |
| rs10036896 | 5 | 132449486 | T | C | 0.2049 | 0.0467 | 0.0072 |
| rs6839086 | 4 | 7869528 | T | G | 0.8263 | −0.0584 | 0.0095 |
| rs13143549 | 4 | 66944813 | G | A | 0.4012 | −0.0391 | 0.0064 |
| rs12509944 | 4 | 66936710 | C | T | 0.6164 | −0.0344 | 0.0060 |
| rs4860797 | 4 | 66935416 | A | G | 0.6344 | −0.0367 | 0.0062 |
| rs13141641 | 4 | 144585304 | C | T | 0.4131 | −0.0603 | 0.0060 |
| rs4408914 | 4 | 144324990 | T | G | 0.7095 | −0.0372 | 0.0064 |
| rs769671 | 4 | 139962464 | T | C | 0.3154 | −0.0369 | 0.0063 |
| rs879394 | 3 | 168992055 | T | G | 0.2626 | 0.0401 | 0.0066 |
| rs6062899 | 20 | 63348441 | A | G | 0.8188 | −0.0634 | 0.0077 |
| rs75550771 | 2 | 55839380 | G | A | 0.0963 | 0.1269 | 0.0209 |
| rs62201158 | 2 | 228688181 | G | A | 0.0792 | −0.0635 | 0.0112 |
| rs17409597 | 2 | 145287492 | C | T | 0.4556 | 0.0393 | 0.0064 |
| rs7598305 | 2 | 144884850 | T | G | 0.2383 | 0.0386 | 0.0069 |
| rs4851569 | 2 | 102366787 | A | C | 0.3905 | 0.0353 | 0.0059 |
| rs2160203 | 2 | 102344364 | G | A | 0.2254 | −0.0414 | 0.0070 |
| rs383925 | 19 | 54279666 | C | T | 0.6514 | −0.0512 | 0.0077 |
| rs7251570 | 19 | 40835845 | G | A | 0.6141 | 0.0392 | 0.0068 |
| rs7504262 | 18 | 37580180 | A | G | 0.2903 | 0.0349 | 0.0064 |
| rs62065216 | 17 | 40062520 | A | G | 0.4586 | 0.0352 | 0.0062 |
| rs4795400 | 17 | 39910767 | T | C | 0.4124 | −0.0417 | 0.0061 |
| rs11658786 | 17 | 39659646 | A | G | 0.6071 | −0.0343 | 0.0061 |
| rs34898535 | 16 | 31014320 | T | C | 0.5248 | −0.0427 | 0.0076 |
| rs12050525 | 15 | 78783404 | C | T | 0.3202 | 0.0483 | 0.0064 |
| rs11854507 | 15 | 78776779 | G | A | 0.3188 | 0.0534 | 0.0065 |
| rs12905273 | 15 | 78710413 | G | A | 0.2692 | −0.0415 | 0.0070 |
| rs11072790 | 15 | 78699683 | T | C | 0.3133 | 0.0535 | 0.0067 |
| rs11072774 | 15 | 78660355 | T | C | 0.2125 | −0.0503 | 0.0075 |
| rs8040868 | 15 | 78618839 | C | T | 0.3761 | 0.0974 | 0.0060 |
| rs28669908 | 15 | 78617925 | A | C | 0.1946 | −0.0866 | 0.0074 |
| rs7359276 | 15 | 78600319 | T | C | 0.6054 | 0.0799 | 0.0068 |
| rs8042849 | 15 | 78525587 | T | C | 0.6971 | −0.0868 | 0.0064 |
| rs2009746 | 15 | 78461760 | G | A | 0.2784 | 0.0856 | 0.0065 |
| rs2568485 | 15 | 78459772 | T | C | 0.7528 | 0.0712 | 0.0068 |
| rs28523913 | 15 | 77900910 | T | C | 0.2243 | 0.0406 | 0.0073 |
| rs12908092 | 15 | 71380414 | C | T | 0.1540 | 0.0442 | 0.0080 |
| rs7181877 | 15 | 67184397 | A | G | 0.2948 | −0.0470 | 0.0067 |
| rs12232354 | 15 | 49351432 | T | C | 0.0737 | −0.0653 | 0.0114 |
| rs7132778 | 12 | 112503550 | A | C | 0.1692 | 0.0846 | 0.0131 |
| rs12579396 | 12 | 112157010 | T | C | 0.1685 | 0.0813 | 0.0132 |
| rs12231873 | 12 | 111894111 | T | C | 0.1635 | 0.0831 | 0.0127 |
| rs76579145 | 12 | 111629113 | T | C | 0.1654 | 0.0709 | 0.0126 |
| rs75295329 | 12 | 110906817 | T | G | 0.0670 | 0.1160 | 0.0190 |
| rs7130588 | 11 | 76559639 | G | A | 0.2655 | 0.0363 | 0.0066 |
| rs10495098 | 1 | 218342968 | T | G | 0.4101 | 0.0346 | 0.0062 |
| rs2099684 | 1 | 161530340 | G | A | 0.3045 | 0.0422 | 0.0063 |
| rs11205303 | 1 | 149934520 | C | T | 0.3501 | 0.0378 | 0.0062 |

Notes:
CHR represented a chromosome, BP represented a chromosome position; A1 represented the effect allele of the SNP, A2 represented the non-effect allele, and AF1 represented a frequency of the effect allele; BETA represented the effect value of the effect allele, and SE represented a standard deviation of the BETA.

### Example 4

A device for assessing an onset risk of COPD, including a detection unit configured to: detect individual SNP marker loci using high-throughput sequencing.

The device further included a data analysis unit: including a computer storage medium and an analysis medium; the analysis medium includes a data receiving module, a data analysis module, and an output module. The individual SNP locus information obtained in Examples 2 and 3 was input into the data analysis unit. The data analysis module in the data analysis unit determined the effect values and a number of effect alleles according to the detection results of the detection unit corresponding to Table 3, and executed the PRS formula of COPD in Example 1 to obtain the PRS value. The output module compared the PRS value obtained by the data analysis module with the genetic risk stratification table in Table 1 or 2 to determine the individual COPD risk.

The device further included a display connected to the output module. The display presented the SNP information of the detection unit, the PRS of the data analysis module, and the genetic risk stratification results of the output module. The above data were processed by visualization software to allow visual conversion and then output to a terminal display screen.

For example, the genotype of a certain sample at the rs9391855 locus was T/T. After looking up Table 3, it was seen that an effect allele T of this SNP locus had an effect value of −0.1705, that is, β=0.1705, G=2. By analogy, a PRS value of the sample could be obtained by multiplying and accumulating the detected effect values of other genotypes and the number of effect alleles. The calculated PRS of the individual was compared with the genetic risk stratification quick-check table shown in Table 1 to obtain the genetic risk stratification to which the individual belonged.

### Example 5

An independent population cohort was validated against 73 SNPs (from Example 2 and Example 3) using the device in Example 4. This independent population cohort was recruited by Beijing Chaoyang Hospital Affiliated to Capital Medical University. 251 COPD patients were recruited from COPD patients undergoing medical examination in the hospital from 2017 to 2022, while 238 control subjects were recruited from the non-COPD population undergoing physical examination in the hospital from 2021 to 2022.

Each of the individuals was calculated based on the SNP information and PRS scoring formula to obtain the individual's PRS. The ROC curve was calculated using the R software packages pROC and ggplot2 and displayed. Individual PRS values were sorted from low to high and divided equally into five groups, and the number of disease and controls in each group was calculated as well as the odds ratio between groups. The results were shown in FIGS. 3A-3C, where FIG. 3A was the ROC curve of an accuracy of the device in Example 4 in predicting COPD in the population. The abscissa represented specificity, and the ordinate represented sensitivity. The ROC had an AUC of 0.652 and a 95% confidence interval of 0.604 to 0.700. This curve indicated that the PRS scoring system had a desirable prediction effect in the population. FIG. 3B showed that the test population was divided into 5 intervals from low to high according to the PRS value. The first interval was used as a benchmark to check the odds ratio of the proportion of sick people in other intervals relative to the first interval. It was seen from the figure that the odds ratio of the fifth interval with the highest score was about 5.4 times that of the first interval. FIG. 3C showed that the test population was divided into 5 intervals from low to high according to the PRS value, and there was a proportion of sick people and non-sick people in each interval. The 1st interval with the lowest score had a prevalence rate of approximately 30%, while the 5th interval with the highest score had a prevalence rate of approximately 70%.

### Example 6

An independent population cohort (the population cohort was the same as that in Example 4) was verified based on 16 SNPs (the SNPs in Example 2) using the device of Example 4.

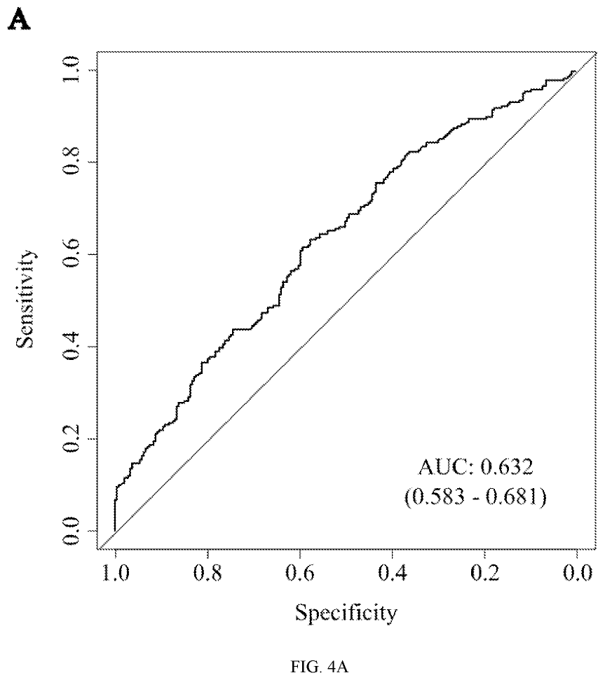

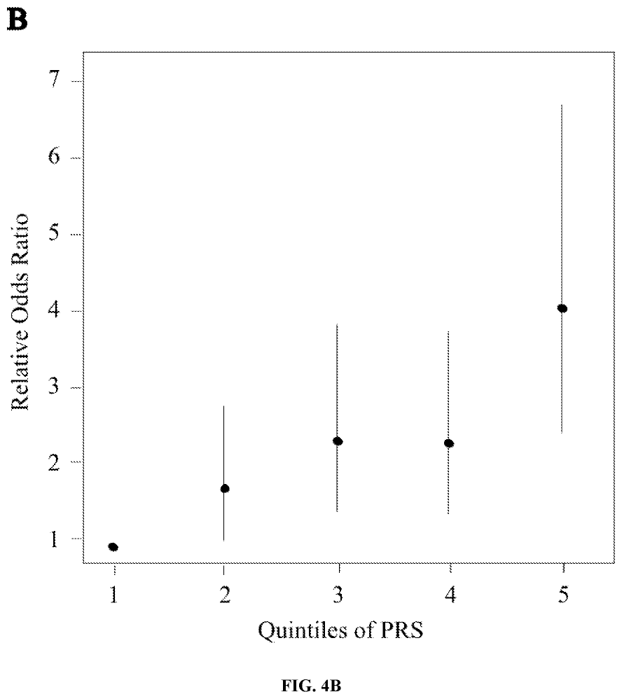

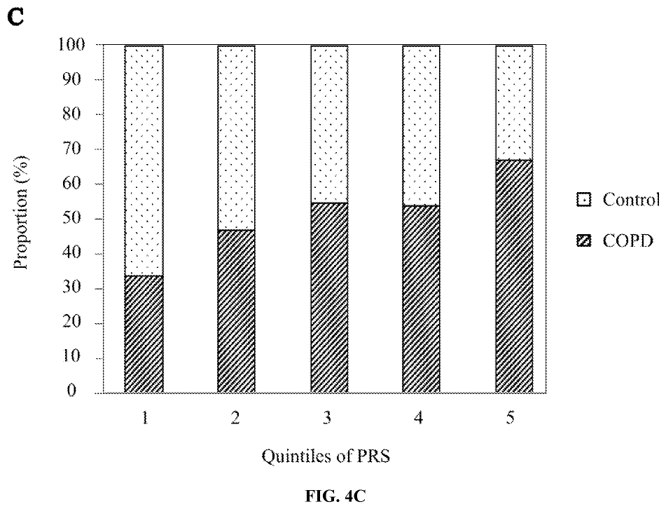

Each of the individuals was calculated based on the SNP information and PRS scoring formula to obtain the individual's PRS. The ROC curve was calculated using the R software packages pROC and ggplot2 and displayed. Individual PRS values were sorted from low to high and divided equally into five groups, and the number of disease and controls in each group was calculated as well as the odds ratio between groups. The results were shown in FIGS. 4A-4C, where FIG. 4A was the ROC curve of an accuracy of the device in Example 4 in predicting COPD in the population. The abscissa represented specificity, and the ordinate represented sensitivity. The ROC had an AUC of 0.632 and a 95% confidence interval of 0.583 to 0.681. This curve indicated that the PRS scoring system had a desirable prediction effect in the population. FIG. 4B showed that the test population was divided into 5 intervals from low to high according to the PRS value. The first interval was used as a benchmark to check the odds ratio of the proportion of sick people in other intervals relative to the first interval. It was seen from the figure that the odds ratio of the fifth interval with the highest score was about 4.1 times that of the first interval. FIG. 4C showed that the test population was divided into 5 intervals from low to high according to the PRS value, and there was a proportion of sick people and non-sick people in each interval. The 1st interval with the lowest score had a prevalence rate of approximately 33%, while the 5th interval with the highest score had a prevalence rate of approximately 67%.

### Example 7

### Use Example

1. An individual to be tested was Wang, Han Chinese, male, 45 years old, smoking. A genetic risk of the subject suffering from COPD was assessed using the device in Example 4 of the present disclosure, and guidance and suggestions were given.

Specifically, fasting blood was collected and DNA in the anticoagulated blood of the individual to be tested was isolated. The DNA was then placed in the detection unit of the device to detect Wang's genotype including the 73 SNP loci mentioned above. The results were as follows: rs11205303:C/C; rs2099684:A/G; rs10495098:T/G; rs75550771:A/A; rs2160203:A/A; rs4851569:C/C; rs7598305:G/T; rs17409597:T/T; rs62201158:A/A; rs1529672:C/C; rs879394:T/T; rs6839086:G/T; rs7676488:T/C; rs4860797:A/A; rs12509944:C/C; rs13143549:A/A; rs1246642:T/T; rs769671:T/T; rs4408914:T/T; rs13141641:T/C; rs2877162:G/T; rs10036896:C/T; rs2057656:C/T; rs11168049:C/C; rs9391855:C/C; rs3104376:C/C; rs9272466:G/G; rs9496212:A/A; rs1931982:T/T; rs1859788:A/G; rs877116:G/G; rs7024396:T/T; rs7130588:A/G; rs141965732:C/C; rs75295329:G/T; rs12229654:T/G; rs79105258:A/A; rs76579145:T/T; rs11066015:A/A; rs12231873:T/T; rs11066132:T/T; rs12579396:T/T; rs77768175:G/G; rs7132778:A/A; rs7295442:G/G; rs12232354:C/C; rs7181877:A/G; rs12908092:T/C; rs28523913:T/T; rs2568485:T/T; rs2009746:A/A; rs8042849:T/T; rs2456020:C/C; rs7359276:C/T; rs28669908:C/C; rs8040868:T/C; rs11072774:C/C; rs11072790:C/C; rs12905273:A/G; rs11854507:A/A; rs12050525:T/T; rs34898535:T/T; rs11658786:A/A; rs4795400:T/T; rs62065216:A/A; rs7504262:G/G; rs2288450:C/C; rs11878604:T/C; rs7251570:A/G; rs4803402:A/A; rs2161245:G/A; rs383925:T/T; and rs6062899:A/G.

The values corresponding to each SNP were obtained by looking up Table 3. The GRS of Wang's COPD was calculated through the data analysis unit using the PRS calculation formula to be 0.0178371. Looking at Table 2, Wang was at high genetic risk for COPD among the population. It was recommended to strictly strengthen and develop good lifestyle and behavioral habits, such as quitting smoking, increasing aerobic activities, and eating healthily. It was also recommended to have a physical examination at least once a year, monitor lung function, and promptly treat any abnormalities.
2. An individual to be tested, Li, was a Han Chinese, female, 50 years old, with a family history of COPD and a long-term history of passive smoking. A genetic risk of the subject suffering from COPD was assessed using the device for assessing PRS of COPD in Example 4, and guidance and suggestions were given.

Li's blood DNA was extracted and the genotype of 73 SNPs was detected. The test results were as follows: rs11205303:C/T; rs2099684:A/G; rs10495098:T/T; rs75550771:A/A; rs2160203:A/A; rs4851569:C/C; rs7598305:G/T; rs17409597:C/C; rs62201158:A/A; rs1529672:C/A; rs879394:T/G; rs6839086:G/G; rs7676488:T/T; rs4860797:A/A; rs12509944:T/T; rs13143549:A/A; rs1246642:T/T; rs769671:T/C; rs4408914:G/G; rs13141641:T/T; rs2877162:G/G; rs10036896:C/C; rs2057656:C/T; rs11168049:C/T; rs9391855:C/T; rs3104376:C/C; rs9272466:G/T; rs9496212:A/A; rs1931982:T/T; rs1859788:A/G; rs877116:G/G; rs7024396:T/T; rs7130588:A/A; rs141965732:C/C; rs75295329:G/G; rs12229654:T/G; rs79105258:A/C; rs76579145:T/C; rs11066015:A/G; rs12231873:T/C; rs11066132:T/C; rs12579396:T/C; rs77768175:G/A; rs7132778:A/C; rs7295442:G/T; rs12232354:C/C; rs7181877:A/G; rs12908092:T/T; rs28523913:T/C; rs2568485:T/T; rs2009746:A/G; rs8042849:T/C; rs2456020:C/C; rs7359276:C/T; rs28669908:C/C; rs8040868:T/C; rs11072774:C/C; rs11072790:C/C; rs12905273:A/G; rs11854507:A/A; rs12050525:T/T; rs34898535:T/T; rs11658786:A/A; rs4795400:T/C; rs62065216:A/G; rs7504262:G/G; rs2288450:C/C; rs11878604:T/C; rs7251570:A/A; rs4803402:A/G; rs2161245:G/G; rs383925:T/T; and rs6062899:A/G.

The values corresponding to each SNP were obtained by looking up Table 3. The GRS of Li's COPD was calculated through the data analysis unit using the PRS calculation formula to be 0.00794495. Comparing Table 2, the results showed that Li was at high genetic risk for COPD among the population. It is recommended that she should avoid passive smoking, strictly maintain good living habits, eat healthily, and do regular aerobic exercise. It was also recommended to have a physical examination at least once a year, monitor lung function, and promptly treat any abnormalities.

The above are only the examples of the present disclosure and therefore do not limit the patent scope of the present disclosure. Any equivalent structure or equivalent process transformation used according to the contents of the specification in the present disclosure, no matter whether it is directly or indirectly used in any other related technical field, should be included within the scope of patent protection of the present disclosure.

1. A single nucleotide polymorphism (SNP) marker for a polygenic risk score (PRS) of chronic obstructive pulmonary disease (COPD), comprising any one or more of the following SNPs: rs9391855, rs1246642, rs7676488, rs1529672, rs2161245, rs4803402, rs11878604, rs2288450, rs2456020, rs7295442, rs77768175, rs11066132, rs11066015, rs79105258, rs12229654, and rs141965732.

2. An SNP marker for a PRS of COPD, comprising any one or more of the following SNPs in addition to the SNP marker according to claim 1: rs7024396, rs877116, rs1859788, rs9272466, rs3104376, rs1931982, rs9496212, rs2877162, rs11168049, rs2057656, rs10036896, rs6839086, rs13143549, rs12509944, rs4860797, rs13141641, rs4408914, rs769671, rs879394, rs6062899, rs75550771, rs62201158, rs17409597, rs7598305, rs4851569, rs2160203, rs383925, rs7251570, rs7504262, rs62065216, rs4795400, rs11658786, rs34898535, rs12050525, rs11854507, rs12905273, rs11072790, rs11072774, rs8040868, rs28669908, rs7359276, rs8042849, rs2009746, rs2568485, rs28523913, rs12908092, rs7181877, rs12232354, rs7132778, rs12579396, rs12231873, rs76579145, rs75295329, rs7130588, rs10495098, rs2099684, and rs11205303.

3. A method of use of the SNP marker according to claim 1 in preparation of a product for assessing an individual COPD risk.

4. A method of use of the SNP marker according to claim 2 in preparation of a product for assessing an individual COPD risk.

5. The method according to claim 3, wherein a PRS value is calculated according to a PRS formula of the COPD, and the PRS value is compared with a genetic risk stratification table to determine the individual COPD risk;
the PRS formula of the COPD is:
$${PRS}_{j} = ( {\sum }_{i}^{N} {β}_{i} \times {G}_{ij} ) / ( P \times {M}_{j} )$$
PRSⱼ represents the PRS of a j-th tested individual; N represents a total number of the SNPs included in PRS calculation; βᵢ represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j;
the genetic risk stratification table is:

| | | | | | |
|---|---|---|---|---|---|
| PRS | <−0.0205 | −0.0205 | −0.0132 | −0.005 to | >0.0136 |
| | | to −0.0132 | to −0.005 | 0.0136 | |
| Risk grouping | Low | Mid-low | Moderate | Mid-high | High. |

6. The method according to claim 4, wherein a PRS value is calculated according to a PRS formula of the COPD, and the PRS value is compared with a genetic risk stratification table to determine the individual COPD risk;
the PRS formula of the COPD is:
$${PRS}_{j} = ( {\sum }_{i}^{N} {β}_{i} \times {G}_{ij} ) / ( P \times {M}_{j} )$$
PRSⱼ represents the PRS of a j-th tested individual; N represents a total number of the SNPs included in PRS calculation; βᵢ represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j;
the genetic risk stratification table is:

| | | | | | |
|---|---|---|---|---|---|
| PRS | <0.0054 | 0.0054 | 0.003 to 0 | 0 to | >0.0043 |
| | | to 0.003 | | 0.0043 | |
| Risk grouping | Low | Mid-low | Moderate | Mid-high | High. |

7. The method according to claim 3, wherein the individual is an individual from an East Asian population.

8. The method according to claim 4, wherein the individual is an individual from an East Asian population.

9. The method according to claim 5, wherein the individual is an individual from an East Asian population.

10. The method according to claim 6, wherein the individual is an individual from an East Asian population.

11. A device for assessing an onset risk of COPD, comprising a detection unit, a data analysis unit, and a display; wherein the detection unit is configured to detect locus information of the SNP marker according to claim 1 in an individual.

12. A device for assessing an onset risk of COPD, comprising a detection unit, a data analysis unit, and a display; wherein the detection unit is configured to detect locus information of the SNP marker according to claim 2 in an individual.

13. The device according to claim 11, wherein the detection unit detects the SNP information of a sample to be tested by the following methods: multiplex PCR-targeted amplicon sequencing, MassARRAY nucleic acid mass spectrometry, customized SNP chip detection, or high-throughput sequencing.

14. The device according to claim 12, wherein the detection unit detects the SNP information of a sample to be tested by the following methods: multiplex PCR-targeted amplicon sequencing, MassARRAY nucleic acid mass spectrometry, customized SNP chip detection, or high-throughput sequencing.

15. The device according to claim 11, wherein the data analysis unit comprises a data receiving module, a data analysis module, and an output module; the data analysis module is configured to determine an effect value and a number of effect alleles according to a detection result of the detection unit corresponding to the following table, and then execute a PRS formula of the COPD to obtain the PRS value, the PRS formula of the COPD is:
$${PRS}_{j} = ( {\sum }_{i}^{N} {β}_{i} \times {G}_{ij} ) / ( P \times {M}_{j} )$$
PRSⱼ represents the PRS of a j-th tested individual; N represents a total number of the SNPs included in PRS calculation; βᵢ represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j;

| | | Chromo- | | Non- | Effect value |
|---|---|---|---|---|---|
| | Chromo- | some | Effect | effect | of the effect |
| SNP | some | position | allele | allele | allele |
| rs9391855 | 6 | 32182024 | T | C | −0.1705 |
| rs1246642 | 4 | 88943295 | T | C | 0.1446 |
| rs7676488 | 4 | 7889408 | T | C | 0.1131 |
| rs1529672 | 3 | 25479091 | A | C | −0.1456 |
| rs2161245 | 19 | 40915612 | A | G | 0.1852 |
| rs4803402 | 19 | 40906280 | A | G | 0.1279 |
| rs11878604 | 19 | 40827379 | T | C | 0.1319 |
| rs2288450 | 19 | 40703272 | T | C | −0.1908 |
| rs2456020 | 15 | 78576056 | T | C | −0.1402 |
| rs7295442 | 12 | 112548943 | T | G | −0.1386 |
| rs77768175 | 12 | 112298314 | A | G | −0.2109 |
| rs11066132 | 12 | 112030402 | T | C | 0.2117 |
| rs11066015 | 12 | 111730205 | A | G | 0.2036 |
| rs79105258 | 12 | 111280427 | A | C | 0.1848 |
| rs12229654 | 12 | 110976657 | T | G | −0.1852 |
| rs141965732 | 12 | 110144533 | T | C | 0.1961 |
| rs7024396 | 9 | 125892044 | T | C | 0.0482 |
| rs877116 | 8 | 10855435 | T | G | −0.0366 |
| rs1859788 | 7 | 100374211 | G | A | −0.0391 |
| rs9272466 | 6 | 32637919 | T | G | 0.0766 |
| rs3104376 | 6 | 32632226 | C | T | 0.0429 |
| rs1931982 | 6 | 142235574 | T | C | −0.0363 |
| rs9496212 | 6 | 141978208 | A | G | −0.0381 |
| rs2877162 | 5 | 44415290 | G | T | 0.0411 |
| rs11168049 | 5 | 148475701 | C | T | −0.0337 |
| rs2057656 | 5 | 132473613 | C | T | −0.0469 |
| rs10036896 | 5 | 132449486 | T | C | 0.0467 |
| rs6839086 | 4 | 7869528 | T | G | −0.0584 |
| rs13143549 | 4 | 66944813 | G | A | −0.0391 |
| rs12509944 | 4 | 66936710 | C | T | −0.0344 |
| rs4860797 | 4 | 66935416 | A | G | −0.0367 |
| rs13141641 | 4 | 144585304 | C | T | −0.0603 |
| rs4408914 | 4 | 144324990 | T | G | −0.0372 |
| rs769671 | 4 | 139962464 | T | C | −0.0369 |
| rs879394 | 3 | 168992055 | T | G | 0.0401 |
| rs6062899 | 20 | 63348441 | A | G | −0.0634 |
| rs75550771 | 2 | 55839380 | G | A | 0.1269 |
| rs62201158 | 2 | 228688181 | G | A | −0.0635 |
| rs17409597 | 2 | 145287492 | C | T | 0.0393 |
| rs7598305 | 2 | 144884850 | T | G | 0.0386 |
| rs4851569 | 2 | 102366787 | A | C | 0.0353 |
| rs2160203 | 2 | 102344364 | G | A | −0.0414 |
| rs383925 | 19 | 54279666 | C | T | −0.0512 |
| rs7251570 | 19 | 40835845 | G | A | 0.0392 |
| rs7504262 | 18 | 37580180 | A | G | 0.0349 |
| rs62065216 | 17 | 40062520 | A | G | 0.0352 |
| rs4795400 | 17 | 39910767 | T | C | −0.0417 |
| rs11658786 | 17 | 39659646 | A | G | −0.0343 |
| rs34898535 | 16 | 31014320 | T | C | −0.0427 |
| rs12050525 | 15 | 78783404 | C | T | 0.0483 |
| rs11854507 | 15 | 78776779 | G | A | 0.0534 |
| rs12905273 | 15 | 78710413 | G | A | −0.0415 |
| rs11072790 | 15 | 78699683 | T | C | 0.0535 |
| rs11072774 | 15 | 78660355 | T | C | −0.0503 |
| rs8040868 | 15 | 78618839 | C | T | 0.0974 |
| rs28669908 | 15 | 78617925 | A | C | −0.0866 |
| rs7359276 | 15 | 78600319 | T | C | 0.0799 |
| rs8042849 | 15 | 78525587 | T | C | −0.0868 |
| rs2009746 | 15 | 78461760 | G | A | 0.0856 |
| rs2568485 | 15 | 78459772 | T | C | 0.0712 |
| rs28523913 | 15 | 77900910 | T | C | 0.0406 |
| rs12908092 | 15 | 71380414 | C | T | 0.0442 |
| rs7181877 | 15 | 67184397 | A | G | −0.0470 |
| rs12232354 | 15 | 49351432 | T | C | −0.0653 |
| rs7132778 | 12 | 112503550 | A | C | 0.0846 |
| rs12579396 | 12 | 112157010 | T | C | 0.0813 |
| rs12231873 | 12 | 111894111 | T | C | 0.0831 |
| rs76579145 | 12 | 111629113 | T | C | 0.0709 |
| rs75295329 | 12 | 110906817 | T | G | 0.1160 |
| rs7130588 | 11 | 76559639 | G | A | 0.0363 |
| rs10495098 | 1 | 218342968 | T | G | 0.0346 |
| rs2099684 | 1 | 161530340 | G | A | 0.0422 |
| rs11205303 | 1 | 149934520 | C | T | 0.0378. |

16. The device according to claim 12, wherein the data analysis unit comprises a data receiving module, a data analysis module, and an output module; the data analysis module is configured to determine an effect value and a number of effect alleles according to a detection result of the detection unit corresponding to the following table, and then execute a PRS formula of the COPD to obtain the PRS value, the PRS formula of the COPD is:
$${PRS}_{j} = ( {\sum }_{i}^{N} {β}_{i} \times {G}_{ij} ) / ( P \times {M}_{j} )$$
PRSⱼ represents the PRS of a j-th tested individual; N represents a total number of the SNPs included in PRS calculation; βᵢ represents an effect value of an effect allele of an i-th SNP; i ranges from 1 to 73; Gᵢⱼ represents a number of the effect alleles of the i-th SNP carried by an individual j, and is 0, 1, or 2; P represents a chromosome ploidy and is 2 in the PRS formula; and Mⱼ represents a number of non-missing SNPs among N SNPs detected in the individual j;

| | | Chromo- | | Non- | Effect value |
|---|---|---|---|---|---|
| | Chromo- | some | Effect | effect | of the effect |
| SNP | some | position | allele | allele | allele |
| rs9391855 | 6 | 32182024 | T | C | −0.1705 |
| rs1246642 | 4 | 88943295 | T | C | 0.1446 |
| rs7676488 | 4 | 7889408 | T | C | 0.1131 |
| rs1529672 | 3 | 25479091 | A | C | −0.1456 |
| rs2161245 | 19 | 40915612 | A | G | 0.1852 |
| rs4803402 | 19 | 40906280 | A | G | 0.1279 |
| rs11878604 | 19 | 40827379 | T | C | 0.1319 |
| rs2288450 | 19 | 40703272 | T | C | −0.1908 |
| rs2456020 | 15 | 78576056 | T | C | −0.1402 |
| rs7295442 | 12 | 112548943 | T | G | −0.1386 |
| rs77768175 | 12 | 112298314 | A | G | −0.2109 |
| rs11066132 | 12 | 112030402 | T | C | 0.2117 |
| rs11066015 | 12 | 111730205 | A | G | 0.2036 |
| rs79105258 | 12 | 111280427 | A | C | 0.1848 |
| rs12229654 | 12 | 110976657 | T | G | −0.1852 |
| rs141965732 | 12 | 110144533 | T | C | 0.1961 |
| rs7024396 | 9 | 125892044 | T | C | 0.0482 |
| rs877116 | 8 | 10855435 | T | G | −0.0366 |
| rs1859788 | 7 | 100374211 | G | A | −0.0391 |
| rs9272466 | 6 | 32637919 | T | G | 0.0766 |
| rs3104376 | 6 | 32632226 | C | T | 0.0429 |
| rs1931982 | 6 | 142235574 | T | C | −0.0363 |
| rs9496212 | 6 | 141978208 | A | G | −0.0381 |
| rs2877162 | 5 | 44415290 | G | T | 0.0411 |
| rs11168049 | 5 | 148475701 | C | T | −0.0337 |
| rs2057656 | 5 | 132473613 | C | T | −0.0469 |
| rs10036896 | 5 | 132449486 | T | C | 0.0467 |
| rs6839086 | 4 | 7869528 | T | G | −0.0584 |
| rs13143549 | 4 | 66944813 | G | A | −0.0391 |
| rs12509944 | 4 | 66936710 | C | T | −0.0344 |
| rs4860797 | 4 | 66935416 | A | G | −0.0367 |
| rs13141641 | 4 | 144585304 | C | T | −0.0603 |
| rs4408914 | 4 | 144324990 | T | G | −0.0372 |
| rs769671 | 4 | 139962464 | T | C | −0.0369 |
| rs879394 | 3 | 168992055 | T | G | 0.0401 |
| rs6062899 | 20 | 63348441 | A | G | −0.0634 |
| rs75550771 | 2 | 55839380 | G | A | 0.1269 |
| rs62201158 | 2 | 228688181 | G | A | −0.0635 |
| rs17409597 | 2 | 145287492 | C | T | 0.0393 |
| rs7598305 | 2 | 144884850 | T | G | 0.0386 |
| rs4851569 | 2 | 102366787 | A | C | 0.0353 |
| rs2160203 | 2 | 102344364 | G | A | −0.0414 |
| rs383925 | 19 | 54279666 | C | T | −0.0512 |
| rs7251570 | 19 | 40835845 | G | A | 0.0392 |
| rs7504262 | 18 | 37580180 | A | G | 0.0349 |
| rs62065216 | 17 | 40062520 | A | G | 0.0352 |
| rs4795400 | 17 | 39910767 | T | C | −0.0417 |
| rs11658786 | 17 | 39659646 | A | G | −0.0343 |
| rs34898535 | 16 | 31014320 | T | C | −0.0427 |
| rs12050525 | 15 | 78783404 | C | T | 0.0483 |
| rs11854507 | 15 | 78776779 | G | A | 0.0534 |
| rs12905273 | 15 | 78710413 | G | A | −0.0415 |
| rs11072790 | 15 | 78699683 | T | C | 0.0535 |
| rs11072774 | 15 | 78660355 | T | C | −0.0503 |
| rs8040868 | 15 | 78618839 | C | T | 0.0974 |
| rs28669908 | 15 | 78617925 | A | C | −0.0866 |
| rs7359276 | 15 | 78600319 | T | C | 0.0799 |
| rs8042849 | 15 | 78525587 | T | C | −0.0868 |
| rs2009746 | 15 | 78461760 | G | A | 0.0856 |
| rs2568485 | 15 | 78459772 | T | C | 0.0712 |
| rs28523913 | 15 | 77900910 | T | C | 0.0406 |
| rs12908092 | 15 | 71380414 | C | T | 0.0442 |
| rs7181877 | 15 | 67184397 | A | G | −0.0470 |
| rs12232354 | 15 | 49351432 | T | C | −0.0653 |
| rs7132778 | 12 | 112503550 | A | C | 0.0846 |
| rs12579396 | 12 | 112157010 | T | C | 0.0813 |
| rs12231873 | 12 | 111894111 | T | C | 0.0831 |
| rs76579145 | 12 | 111629113 | T | C | 0.0709 |
| rs75295329 | 12 | 110906817 | T | G | 0.1160 |
| rs7130588 | 11 | 76559639 | G | A | 0.0363 |
| rs10495098 | 1 | 218342968 | T | G | 0.0346 |
| rs2099684 | 1 | 161530340 | G | A | 0.0422 |
| rs11205303 | 1 | 149934520 | C | T | 0.0378. |

17. The device according to claim 15, wherein the output module is configured to compare the PRS value obtained by the data analysis module with a genetic risk stratification table, and then determine the individual COPD risk, the genetic risk stratification table is:

| | | | | | |
|---|---|---|---|---|---|
| PRS | <−0.0205 | −0.0205 | −0.0132 | −0.005 to | >0.0136 |
| | | to −0.0132 | to −0.005 | 0.0136 | |
| Risk grouping | Low | Mid-low | Moderate | Mid-high | High. |

18. The device according to claim 16, wherein the output module is configured to compare the PRS value obtained by the data analysis module with a genetic risk stratification table, and then determine the individual COPD risk, the genetic risk stratification table is:

| | | | | | |
|---|---|---|---|---|---|
| PRS | <0.0054 | 0.0054 to | 0.003 to 0 | 0 to | >0.0043 |
| | | 0.003 | | 0.0043 | |
| Risk grouping | Low | Mid-low | Moderate | Mid-high | High. |

19. The device according to claim 15, wherein the display is connected to the output module and then configured to display results of the detection unit, the data analysis module, and the output module.

20. The device according to claim 16, wherein the display is connected to the output module and then configured to display results of the detection unit, the data analysis module, and the output module.
